(19) United States
(12) Patent Application Publication
(10) Pub. No.: US 2024/0156498 A1
(43) Pub. Date: May 16, 2024

(54) BONE PLATE SYSTEM

(71) Applicant: Xtant Medical Holdings, Inc., Belgrade, MT (US)

(72) Inventors: Scott J. Perrow, Ishpeming, MI (US); Michael D. Kakuk, Skandia, MI (US)

(21) Appl. No.: 18/417,139

(22) Filed: Jan. 19, 2024

Related U.S. Application Data
(62) Division of application No. 17/211,489, filed on Mar. 24, 2021, now Pat. No. 11,877,779.
(60) Provisional application No. 63/000,163, filed on Mar. 26, 2020.

(51) Int. Cl. A61B 17/80

(52) U.S. Cl. CPC A61B 17/8047; A61B 17/8888

(57) ABSTRACT

In one aspect, a bone plate system is provided that includes a bone plate body having a throughbore and a bone screw. The bone plate system includes a slider slidably connected to the bone plate body to slide between a clearance position wherein the slider permits a bone screw head portion to be advanced into the throughbore and seated against the bone plate body and an interference position wherein the slider inhibits bone screw back out from the throughbore. The bone plate system includes a resilient pin and the slider has a surface configured to deflect the resilient pin as the bone screw head portion shifts the slider from the interference position toward the clearance position. The deflected resilient pin urges the slider from the clearance position toward the interference position upon the bone anchor head portion seating against the bone plate body in the throughbore.

### CROSS-REFERENCE TO RELATED APPLICATION

This application claims the benefit of U.S. Provisional Patent Application No. 63/000,163, filed Mar. 26, 2020, which is hereby incorporated by reference in its entirety.

### FIELD

This disclosure relates to bone plate systems and, more specifically, to bone plate systems having devices to limit back-out of bone screws of the bone plate systems.

### BACKGROUND

Bone plate systems are often used to stabilize adjacent bones or portions of a bone. For convenience, the term “bone” as used herein is intended to encompass a bone or a portion of a bone. Bone plate systems often include a bone plate which is placed against the bones and bone screws that are driven into throughbores of the bone plate and into the bone to secure the bone plate to the bones. Bone plate systems often include devices to inhibit back-out of the bone screws to ensure the bone plate and bone screw construct remains secured with the bones. The devices also limit back-out of the bone screw to keep the bone screws from projecting outward from the bone plate and irritating surrounding tissues.

Bone plates are often used to stabilize vertebrae to address an injury, intervertebral disc replacement, or other situation. Some bone plate systems for the cervical region of the spine utilize a spring retainer in each throughbore of the bone plate that deflects out of the way of the bone screw to permit a bone screw to advance into the throughbore and returns to its initial position in the throughbore to inhibit back-out the bone screw. The spring retainer may be made of a wire that extends across an upper surface of the bone screw head to limit back-out once the bone screw head is seated in the throughbore. One issue with these spring retainers is that the wire material is very thin which may make it difficult for a surgeon to visually ascertain whether the retainer is positioned above the bone screw head to limit back-out.

Some bone plate systems have a multiple-stage bone screw installation process. In a first stage, a bone screw is driven into a throughbore. In a subsequent stage, the surgeon moves a rigid member into an overlapping position with the bone screw head to limit back-out of the bone screw. This multiple-stage process may complete installation of a bone plate system.

### BRIEF DESCRIPTION OF THE DRAWINGS

FIG. 1 is a perspective view of a bone plate system including a bone plate, bone screws, and sliders of the bone plate that inhibit back-out of the bone screws from throughbores of the bone plate;

FIG. 2 is a top plan view of the bone plate of FIG. 1 showing the sliders of the throughbores in an interference position wherein the sliders extend into the throughbores to overlap head portions of the bone screws,

FIG. 3 is a cross-sectional view taken across line 3-3 in FIG. 1 showing a resilient pin of the bone plate in an initial configuration wherein ends of the pin are pressed against surfaces of pockets of the bone plate;

FIG. 4 is a cross-sectional view similar to FIG. 3 showing the slider having been shifted radially outward which shifts the end portions of the pin out of contact with the pocket surfaces of the bone plate;

FIG. 5 is a cross-sectional view similar to FIG. 3 showing the slider in a clearance position wherein a bone screw may be driven into the throughbore and seated therein;

FIG. 6 is a cross-sectional view taken across line 6-6 in FIG. 2 showing a wider lower portion of the slider received in an undercut of the bone plate;

FIG. 7 is a cross-sectional view of the bone plate taken across line 7-7 in FIG. 2 in combination with a bone screw being driven into a throughbore and the slider in the interference position thereof;

FIG. 8 is a cross-sectional view similar to FIG. 7 showing a lower surface of a head portion of the bone screw contacting a tapered surface of the slider and beginning to shift the slider radially outward;

FIG. 9 is a cross-sectional view similar to FIG. 7 showing the bone screw advanced farther into the thorughbore and a side surface of the bone screw head portion keeping the slider in the clearance position thereof which permits the bone screw head portion to travel beyond the slider;

FIG. 10 is a cross-sectional view similar to FIG. 7 showing the bone screw head portion seated in the throughbore and the slider having shifted radially inward to the interference position wherein a lip portion of the slider is positioned above the bone screw head portion to inhibit back-out of the bone screw from the throughbore;

FIG. 11 is a perspective view of the resilient pin of FIG. 3 in an unloaded configuration;

FIG. 12 is a perspective view of one of the sliders of the bone plate of FIG. 1 showing a channel for receiving the resilient pin;

FIG. 13 is a side elevational view of the slider of FIG. 12 showing a foot portion and a wall of the slider on opposite sides of the channel;

FIG. 14 is a front elevational view of the slider of FIG. 12 showing the step profile of the sides of the slider;

FIG. 15 is a bottom plan view of the slider of FIG. 12 showing a curved profile of the wall that bends an intermediate portion of the resilient pin;

FIG. 16 is a side elevational view of one of the bone screws of FIG. 1;

FIGS. 17, 18, 19 and 20 are top plan views of a throughbore of the bone plate of FIG. 1 showing an example process for assembling the slider and pin in the bone plate body;

FIG. 21 is a perspective view of a bone screw removal tool for removing a bone screw from a bone plate system of FIG. 1;

FIG. 22 is a perspective view of a distal end of the bone screw remover tool of FIG. 21 showing a driving tip of the tool;

FIG. 23 is a cross-sectional view taken across line 23-23 in FIG. 21 showing an internal shaft of the tool extending through a sleeve of the tool;

FIG. 24 is a cross-sectional view of a distal end of the bone screw remover tool showing the sleeve positioned to shift the slider radially outward with turning of the sleeve.

### DETAILED DESCRIPTION

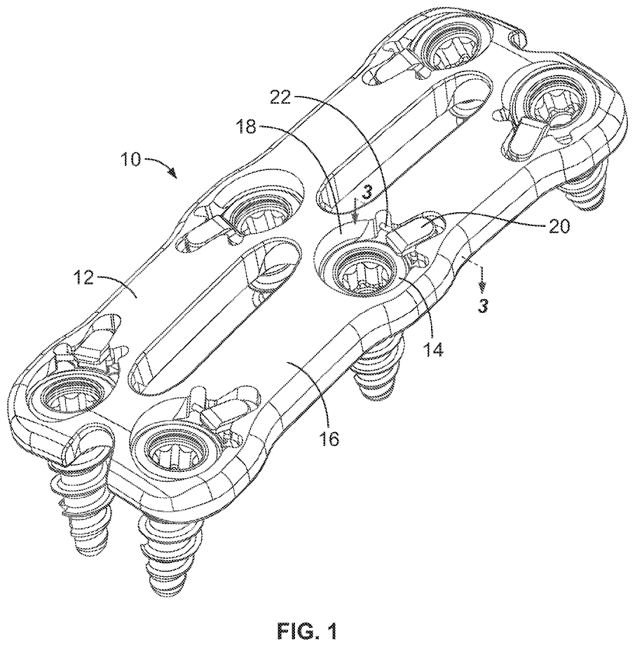

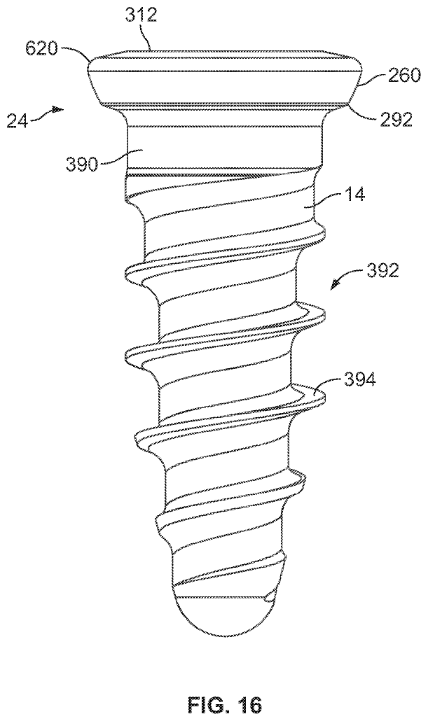

Regarding FIG. 1, a bone plate system 10 is provided that includes a bone plate 12 and bone screws 14. The bone plate 12 includes a body 16 having throughbores 18 formed therein for receiving the bone screws 14. The throughbores 18 each have a central, longitudinal axis 19. The bone plate 12 includes retainers, such as one or more sliders 20, and resilient members, such as pins 22, urging the sliders 20 laterally to an interference position. The bone screws 14 each include a head portion 24 (see FIG. 16) having a tapered surface 260 configured to urge the respective slider 20 from the interference position to a clearance position and permit the head portion 24 to advance beyond the slider 20 and be seated in the throughbore 18. The pin 22 resiliently returns the slider 20 to the interference position above the bone screw head portion 24 to inhibit back-out of the bone screw 14 from the throughbore 18.

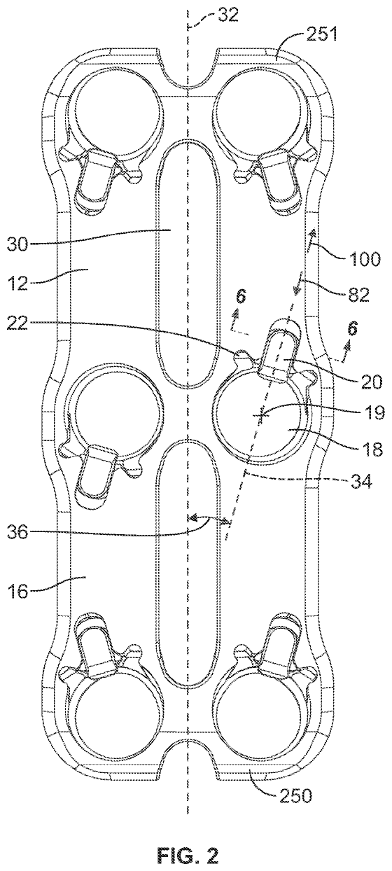

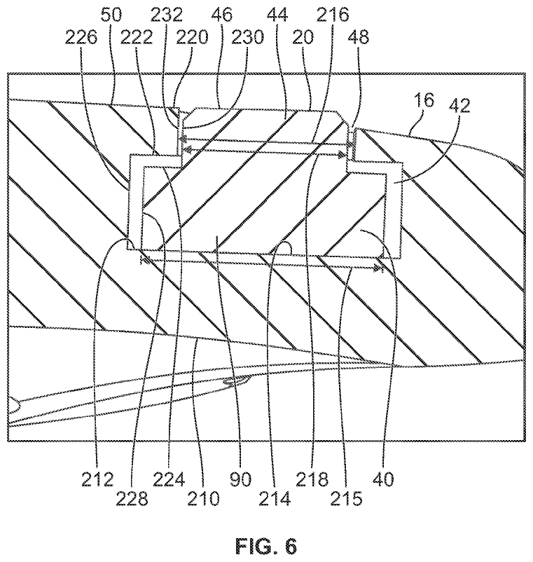

Regarding FIG. 2, the bone plate body 16 includes one or more windows 30 to permit bones or an implant therebetween to be observed by a surgeon. The bone plate body 16 may be elongated and have a longitudinal axis 32 and each slider 20 may be slidable along a path 34. In one embodiment, the path 34 is straight and extends at an angle 36 relative to the longitudinal axis 32. The one plate body 16 may include a receptacle for each slider 20 that includes an undercut 42 and an opening 48. The slider 20 may be constrained to sliding movement in the bone plate body 16 by way of a lower level 40 (see FIG. 6) received in the undercut 42 of the bone plate body 16. The slider 20 has a narrower, upper level 44 with an upper face 46 visible through the opening 48 in an upper surface 50 of the bone plate body 16. The opening 48 opens to the undercut 42 and permits the upper level 44 of the slider 20 to be visible whether the slider 20 is in the interference position (see FIG. 2) or the clearance position (see FIG. 9). In this manner, a surgeon may readily visually ascertain whether the slider 20 has been shifted back by the pin 22 to the interference position upon seating of the bone screw 14 in the throughbore 18. Further, the pin 22 snaps the slider 20 back to the interference position once the bone screw head portion 24 is advanced beyond the slider 20 which provide a tactile indication that the slider 20 has returned to the interference position.

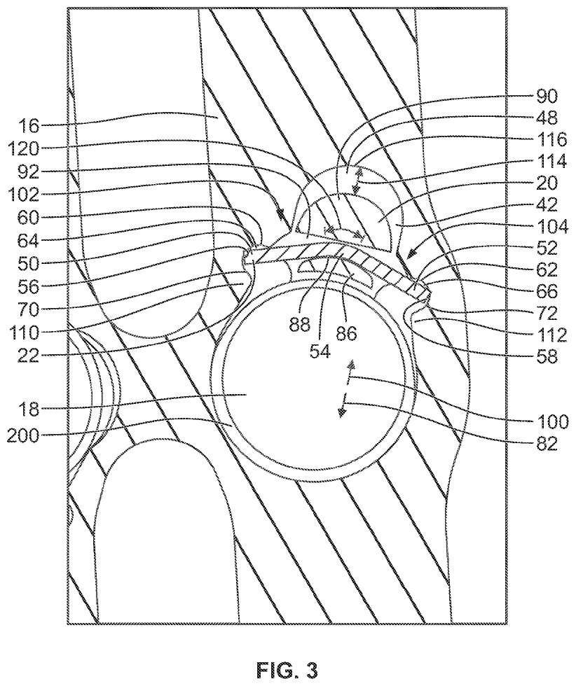

Regarding FIGS. 2 and 3, the slider 20 is shown in the interference position. The pin 22 is under a preload with the pin 22 and slider 20 mounted to the bone plate body 16. Regarding momentarily to FIG. 11, the pin 22 has an underformed configuration wherein the pin 22 is straight. Returning to FIG. 3, the pin 22 has opposite end portions 50, 52, and an intermediate portion 54 therebetween. The pin 22 has ends 56, 58 and a longitudinal axis 59 (see FIG. 11) extending between the ends 56, 58. The pin 22 is loaded upon assembly of pin 22 and slider 20 with the bone plate body 16 as discussed in greater detail below. The pin 22 is bent when the slider 20 is in the interference position and is bent further with the slider 20 in the clearance portion. The pin may have a cross-section transverse to the length 80 that is uniform for at least a majority of the length 80. The cross-section may be, for example, circular or rectangular.

Regarding FIG. 3, the bone plate body 16 has pockets 60, 62 that include recesses 64, 66 which open to the undercut 42. The pockets 60, 62 include surfaces 70, 72 against which the ends 56, 58 are tightly engaged by the preloaded pin 22. The pin 22 is biased to straighten out to its unloaded configuration (see FIG. 11); however, the distance between the surfaces 70, 72 is less than the length 80 of the pin 22 in the unloaded configuration. In other words, the pocket 60, 62 are too close together to permit the pin 22 to straighten out. The engagement between the ends 56, 58 of the pin 22 and the surfaces 70, 72 of pockets 60, 62 inhibits the pin 22 from shifting radially inward in direction 82.

The slider 20 has a bending member, such as a wall 86 having a curved surface 88 configured to contact the intermediate portion 54 of the pin 22. As shown in FIG. 3, the slider 20 is in the initial, interference position wherein the intermediate portion 54 of the pin contacts the wall 86 to bias the slider 20 toward the interference position. The slider 20 further includes a foot portion 90 spaced from the wall 86 to form a channel 92 therebetween. The pin 22 extends in the channel 92 with the end portions 50, 52 of the pin projecting laterally outward from the slider 20 and into the pockets 60, 62. The wall 86 supports the intermediate portion 54 of the pin 22 as a fulcrum about which the pin 22 may bend with shifting of the slider 20 in direction 100 toward the clearance position thereof.

Regarding FIG. 3, the bone plate body 16 includes supports 102, 104 on opposite sides of the slider 20 configured to support the end portions 50, 52 and provide a base for the end portions 50, 52 to contact and urge the slider 20 back toward the interference position. Opposite the supports 102, 104, the bone plate body 16 includes retainer portions 110, 112 that are spaced apart a distance smaller than the length 80 of the pin 22 to inhibit the pin 22 from straightening out and exiting the bone plate body 16.

With the slider 20 in the interference position, the foot portion 90 of the slider 20 is spaced a distance 114 from a wall 116 of the bone plate body 16. Further, the intermediate portion 54 of the pin 22 is in contact with the curved surface 88 a length 120. The curved surface 88 provides a gradual transition for the pin 22 that limits stress risers in the pin 22.

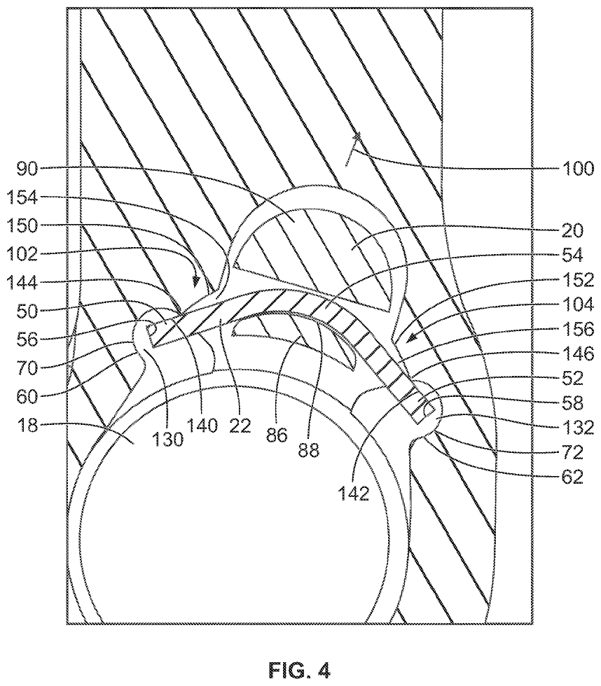

Regarding FIG. 4, the slider 20 has been shifted in radial or lateral outward direction 100 by a bone screw head portion (not shown in FIG. 4) as the bone screw head portion is being advanced into the throughbore 18. The shifting of the slider 20 in direction 100 causes the wall 86 and curved surface 88 thereof to press against a center portion, such as the intermediate portion 54, of the pin 22. The wall 86 shifts the intermediate portion 54 in direction 100 with the slider 20 and shifts the ends 56, 58 of the pin 22 away from the surfaces 70, 72 of the pockets 60, 62. This forms gaps 130, 132 between the ends 56, 58 and the surfaces 70, 72. With the slider 20 in this intermediate position, the ends 56, 58 of the pin 22 are no longer pressed against the surfaces 70, 72. Further, the end portions 50, 52 have opposite side portions, such as outer side surface portions 140, 142, contacting portions of the support 102, 104 such as edges 144, 146. The intermediate portion 54 of the pin 22 is bent more in the intermediate position of FIG. 4 than with the slider 20 in the interference position of FIG. 3. Further, the end portions 50, 52 are closer together when the slider 20 is in the intermediate position than when the slider 20 is in the interference position. As shown in FIG. 4, the supports 102, 104 include support surfaces 150, 152 that are spaced from the end portions 50, 52 of the pin 22 by gaps 154, 156 when the slider 20 is in the intermediate position.

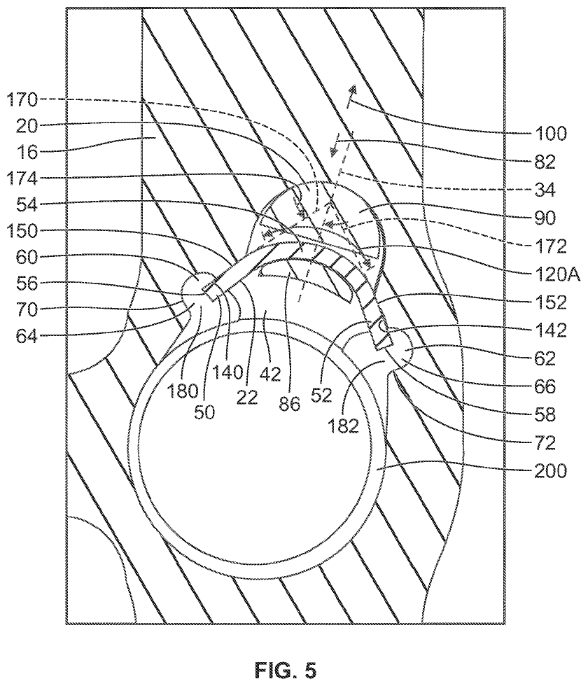

Regarding FIG. 5, the bone screw head portion has shifted to the slider 20 radially outward in direction 100 to the clearance position thereof. This causes the wall surface 88 of the slider 20 to further shift the intermediate portion 54 away from the throughbore 18 so that the pin 22 extends a length 120A along the wall surface 88 that is greater than the length 120 as shown in FIG. 3. The support surfaces 150, 152 of the bone plate body 16 may be curved to compliment the curving of the pin 22 caused by the shifting of the slider 20 to the clearance position. The support surfaces 150, 152 may generally extend along axes 170, 172 that are each oriented at an angle 174 relative to the path 34 of the slider 20. As shown in FIG. 5, the ends 56, 58 of the pin 22 are spaced from the pocket surfaces 70, 72 by larger gaps 180, 182 than in the intermediate position of the slider 20 shown in FIG. 4. The ends 56, 58 have at least been partially withdrawn from the recesses 64, 66 of the pockets 60, 62 when the slider 20 is in the clearance position.

As shown in FIG. 5, the pin 22 generally has three points of contact including the support surface 150, the support surface 152, and the surface 88. The pin 22 is resiliently deformed in this configuration and is biased to straighten out and urge the slider 20 back radially inward in direction 82 toward the interference position once the bone head portion 24 has seated against a seating surface 200 of the bone plate body 16. Once the bone head portion 24 is seated against the seating surface 200, the pin 22 straightens out which causes the intermediate portion 54 to urge the wall 86 of the slider 20 back laterally inward in direction 82 and permits the end portions 50, 52 of the pin 22 to snap back into the pockets 60, 62 and press against the surfaces 70, 72.

Regarding FIG. 6, the bone plate body 16 has a lower surface 210 opposite the upper surface 50. The undercut 42 includes a floor 212 that supports a bottom face 214 of the lower level 40 of the slider 20. The lower level 40 has a width 215 that is wider than a width 216 of the opening 48 and a width 218 of the upper level 44 of the slider 20. The bone plate body 16 includes overhangs, such as upper walls 220, that at least in part define the opening 48 and have lower surfaces 222 facing upper faces 224 of the lower level 40. The bone plate body 16 further includes lower side walls 226 facing side wall portions 228 of the lower level 40. At the upper level 44, the slider 20 includes upper side wall portions 230 that face upper side walls 232 of the upper walls 220. In this manner, the sliders 20 have notched profiles on the opposite sides thereof and the bone plate body 16 has narrow sections that extend into the notched profiles and form an example of a slide connection between the bone plate body 16 and the slider 20.

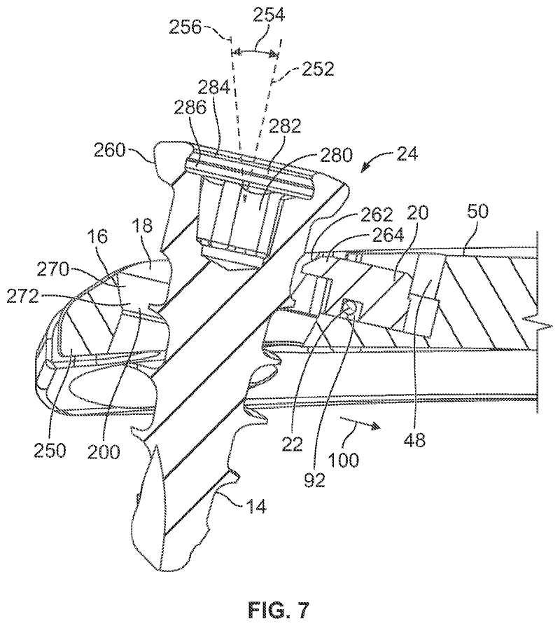

Regarding FIG. 7, a throughbore 18 at an end portion 250 of the bone plate body 16 is shown. The throughbore 18 has a central, longitudinal bore axis 252 that may be at an angle 254 from an axis 256 generally normal to the upper surface 250 of the bone plate body 16. The throughbore 18 may be configured to permit the surgeon to toe out the bone screws 14 in the throughbores 18 at the end portions 250, 251 of the bone plate body 16. The head portion 24 of the bone screw 14 includes a tapered surface 260 that performs multiple functions. One function is to engage a tapered surface 262 of a lip portion 264 of the slider 20 and cammingly shift the slider 20 in lateral or radial direction 100 as the bone head portion 24 is advanced into the throughbore 18. Another function of the surface 260 is to seat against the seating surface 200 of the bone plate body 16. The seating surface 200 may be generally concave, including an upper portion 270 having an inner diameter and a lower portion 272 having a smaller inner diameter. The seating surface 200 permits polyaxial insertion of the bone screw 14 into the throughbore 18. Further, the surfaces 200, 260 may be configured to permit controlled pivoting of the bone screw 14 relative to the bone plate body 16 such as due to subsidence of the bones to which the bone plate 12 is secured.

The head portion 24 of the bone screw 14 includes a rotary drive structure 280 for receiving a driving tool. The head portion 24 may include an opening 282 that opens to the rotary drive structure 280, a collar portion 284, and an undercut 286. The driver may have a portion that extends into the undercut 286 to retain the bone screw 14 on the driver tool as the driver tool is used to advance the bone screw 14 into the throughbore 18. The rotary drive structure 280 may have a Torx configuration as one example.

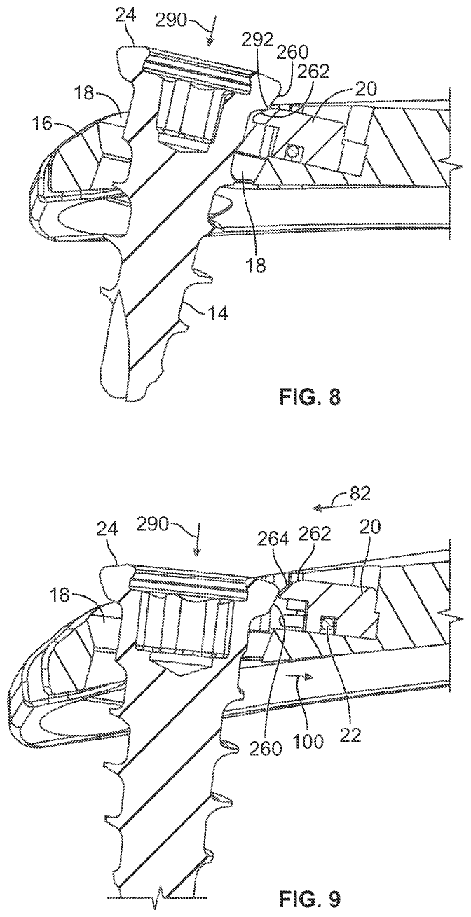

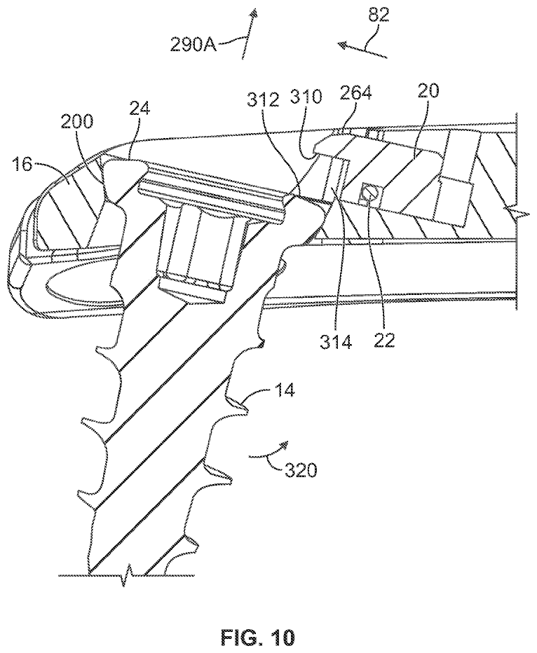

Regarding FIG. 8, the bone screw 14 is shown and driven in direction 290 into the throughbore 18 with a lower edge 292 of the bone screw head portion 24 contacting the tapered surface 262 of the slider 20. Regarding FIG. 9, continued advancing of the bone screw 14 in direction 290 brings the tapered surface 260 of the bone screw head portion 24 into camming engagement with the tapered surface 262 of the slider 20 such that the camming engagement between surfaces 260, 262 shifts the slider 20 outward laterally in direction 100 to the clearance position thereof so that the head portion 24 may be advanced into the throughbore 18. As the head portion 24 is advanced in direction 290, the pin 22 is deflected and biases the slider 20 back toward the interference position in direction 82. Regarding FIG. 10, the head portion 24 of the bone screw 14 shown seated against the seating surface 200 of the bone plate body 16. The pin 22 has urged the slider 20 in direction 82 back to the interference position. The slider 20 has the lip portion 264 thereof with a lower surface 310 overlapping upper surface 312 of the bone screw head portion 24. Thus, the lower surface 310 of the slider 20 is positioned to contact the upper surface 312 and inhibit back-out of the bone screw 14 in direction 290A. The lip portion 264 of the slider 20 provides a gap 314 between the lower surface 310 and the upper surface 312. The gap 314 permits controlled pivoting or angulation of the bone screw 14. Specifically, the gap 314 permits the bone screw 14 to turn in direction 320 with subsidence of the bones stabilized by the bone plate system 10.

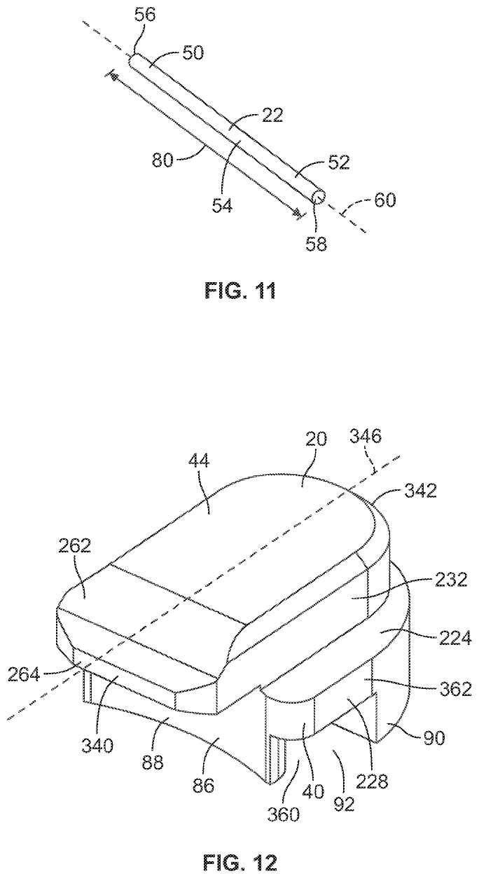

Regarding FIGS. 12 and 14, the slider 20 has an inboard end 340, an outboard end 342, and an axis 346 extending therebetween. The narrower upper level 44 forms a step profile 348 with a notch 350 on opposite sides of the slider 20 for receiving the upper walls of the bone plate body 16. The upper side wall portions 230 may be parallel to the lower side wall portions 228 and the upper face 224 of the lower level 40 extends therebetween.

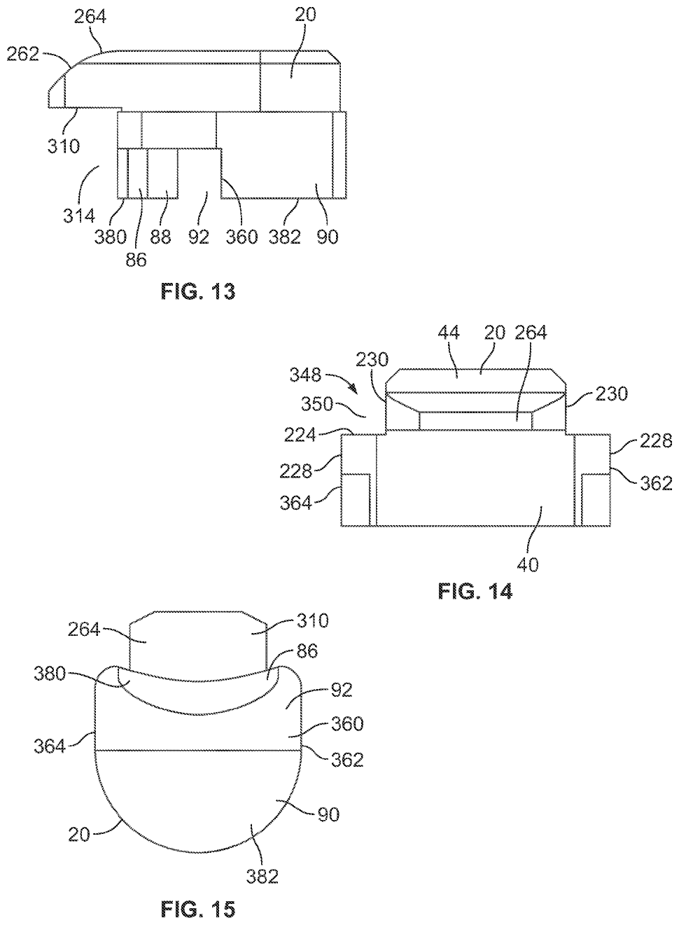

Regarding FIGS. 12 and 13, the slider 20 includes a through opening for receiving the pin 22 such as the channel 92. The channel 92 may include a flared portion 360 at sides 362, 364 of the slider 20. The flared portions 360 provide clearance for the intermediate portion 54 of the pin 22 to deflect as the slider 20 is moved between the clearance and interference positions.

Regarding FIGS. 13 and 15, the wall 86 and the foot portion 90 of the slider 20 include lower surfaces 380, 382 configured to slide along a floor surface 212 (see FIG. 6) of the bone plate body 16. The surfaces 380, 382 and 212 may be flat or have other shapes, e.g. protrusions, or textures.

Regarding FIG. 16, the bone screw 14 includes the head portion 24 having a cylindrical surface 390. The bone screw 14 further includes a shank portion 392 depending from the head portion 24 that may include threads 394. Threads 394 may be single lead or multiple lead threads as some examples.

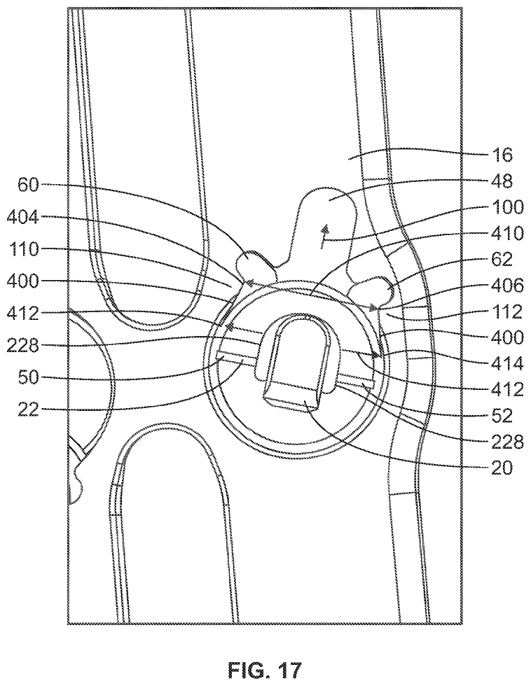

Regarding FIGS. 17-20, a method of assembling the slider 20, pin 22, and the bone plate body 16 is shown. Regarding FIG. 17, the slider 20 is positioned in the throughbore 18 with the pin 22 in a straight, undeflected configuration extending in the channel 92. The end portions 50, 52 of the pin 22 extend outward from the side wall portions 228 of the slider 20. The retainer portions 110, 112 of the bone plate body 16 include tapered guide surfaces 400, 402 that are inclined and extend toward each other as the surfaces 400, 402 extend away from the throughbore 18 and end at edges 404, 406. The edges 404, 406 are separated by a width or distance 410 that is less than a distance 412 of the widest portions 412, 414 of the tapered guide surfaces 400, 402. The distance 410 may be less than a maximum width of the pin 22 such as the length 80.

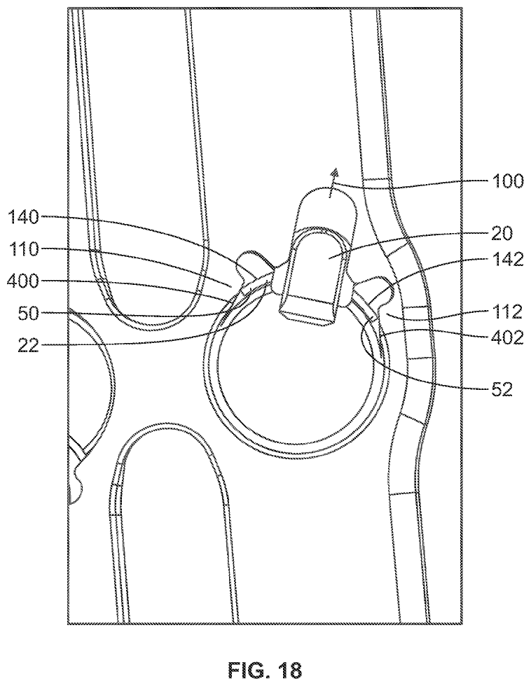

Regarding FIG. 18, the slider 20 has been shifted in direction 100 such that the outer side surface portions 140, 142 of the pin 22 slide along the tapered guide surfaces 400, 402. Because the length 80 of the pin 22 in the undeflected configuration is larger than the distance 410, shifting the slider 20 in direction 100 causes the pin 22 to bend as the intermediate portion 54 of the pin 22 is bent and moves with the slider 20 while the end portions 50, 52 are engaging and sliding along the tapered guide surfaces 400, 402. The tapered guide surfaces 400, 402 cause the end portions 50, 52 to be urged together as the slider 20 shifts in direction 100.

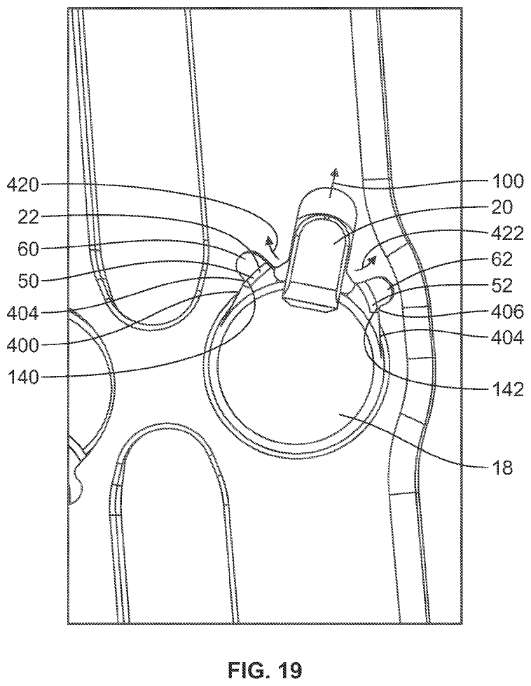

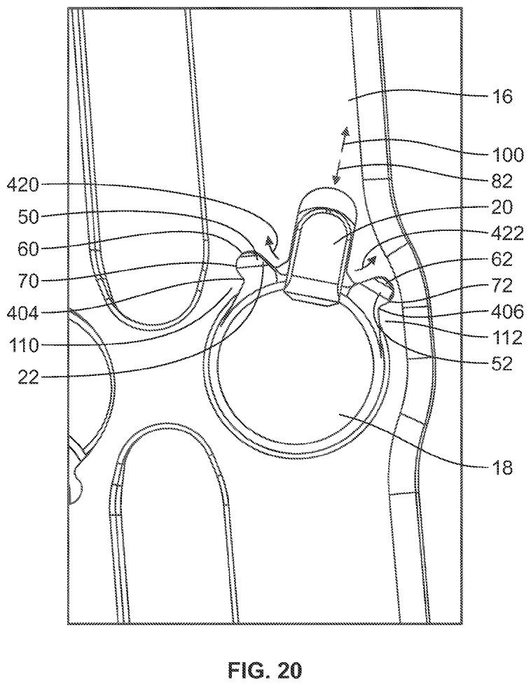

Regarding FIG. 19, the slider 20 is continued to be shifted in direction 100 and the outer side surface portions 140, 142 of the pin 22 are engaged with the edges 404, 406 of the bone plate body 16. The pin 22 is bent and is resiliently urging the end portions 50, 52 apart in directions 420, 422. Regarding FIG. 20, the slider 20 has been shifted in direction 100 to a position where the end portions 50, 52 of the pin 22 have shifted radially beyond edges 404, 406 such that the end portions 50, 52 may snap apart and into the pockets 60, 62. Once the end portions 50, 52 have snapped into the pockets 60, 62, the slider 20 may be released and the pin 22 urges the end portions 50, 52 in directions 420, 422 against the surfaces 70, 72 of the pockets 60, 62. As noted above, the edges 404, 406 are separated by the distance 410 which is less than the length 80 of the unloaded pin 22 such that the retainer portions 110, 112 of the bone plate inhibit the pin 22 from fully unloading and urging the slider in direction 100A. In this manner, the pin 22 may be assembled with the slider 20 and the bone plate body 16 and the preload applied to the pin 22. The preload in the pin 22 keeps the pin 22 in a bent configuration in the bone plate body 16 so that the pin 22 resists movement of the slider 20 in direction 82A and positions the pin 22 to be shifted with the slider 20 in direction 100 upon the advancing of the bone screw into the throughbore 18.

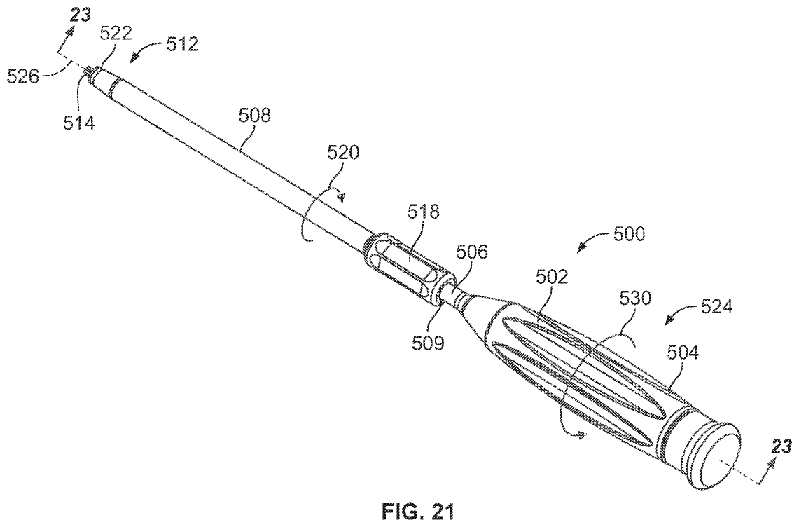

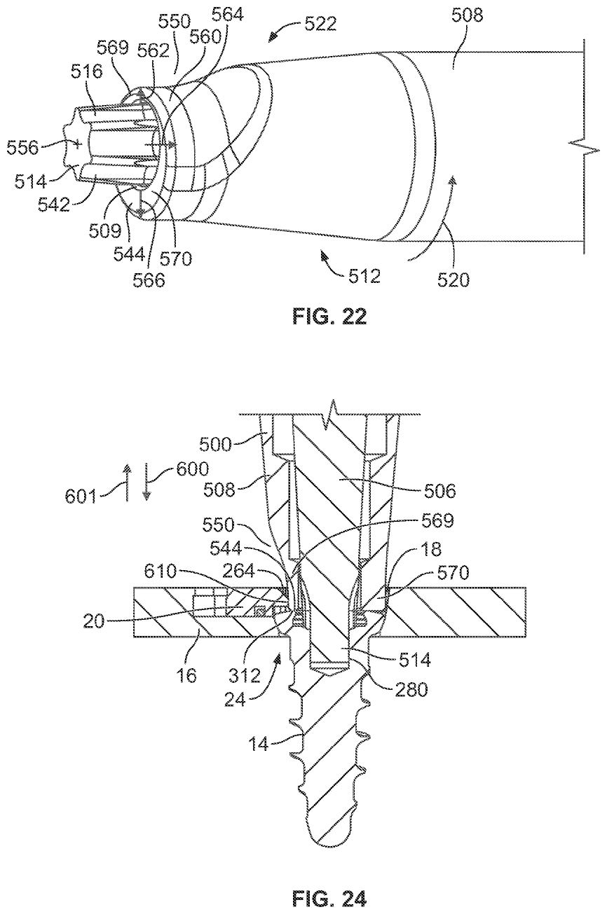

Regarding FIG. 21, a bone screw remover tool 500 is provided that permits a surgeon to connect the bone screw remover tool 500 to a bone screw 14 in a throughbore 18, shift the slider 20 out of the way of the bone screw 14, and then turn the bone screw 14 to remove the bone screw 14 from the throughbore 18. More specifically, the bone screw remover tool 500 includes a driver 502 having a handle 504 and a shaft 506. The bone screw remover tool 500 has a sleeve 508 with a cannula 509 through which the shaft 506 extends until protruding at a driving member, such as a driving tip 514 having a rotary drive structure 516 (see FIG. 22). The sleeve 508 has a knob 518 and is rotatably connected to the shaft 506 of the driver 502. In this manner, the surgeon may turn the knob 518 in direction 520 to cause the sleeve 508 and a cam portion 522 thereof to shift the slider 20 from the interference position to the clearance position and then may turn the handle 504 and driving tip 514 connected thereto to remove the associated bone screw 14. As shown in FIG. 21, the bone screw remover tool 500 has a distal end portion 512 and a proximal end portion 524, and a longitudinal axis 526 extending therebetween. As noted above, the sleeve 508 may be rotated relative to the driver 502 in direction 520. Conversely, the driver 502 may be rotated in direction 530 relative to the sleeve 508 to loosen the bone screw 14 while the sleeve 508 keeps the slider 20 in the clearance position thereof. In some embodiments, a surgeon may connect the driving tip 514 to a bone screw 14, turn the sleeve 508 in direction 530 to shift the slider 20 to the clearance position, and then turn the driver 502 in direction 530 to remove the bone screw 14 from the throughbore 18.

Regarding FIG. 22, the driving tip 514 extends out from the cannula 509 of the sleeve 508 to expose the rotary drive structure 516, such as projections that fit into recesses of the rotary drive structure 280 of the bone screw 14. The rotary drive structure 516 may be, for example, a Torx driver. The driving tip 514 extends out of a distal surface 544 of the sleeve 508. The surface 544 may be adapted to seat against the upper surface 312 (see FIG. 24) of the bone screw head portion 24. The cam portion 522 of the sleeve 508 has a recess 550 that is axially aligned with the slider 20 before the driving tip 514 is advanced into the throughbore 18. The recess 550 provides clearance for the sleeve 508 to be advanced without contacting the lip portion 264 of the slider 20. The surface 544 may have a general circular periphery with a center that is eccentric to a center 556 of the driving tip 514. The cam portion 522 includes a cam surface 560 having radii 562, 564, 566 from the center 556 of the driving tip 514 that increase as the cam surface 560 extends clockwise (in FIG. 22) about the driving tip 514 from the approximate twelve o'clock position to the proximate six o'clock position. Once the driving tip 514 has been advanced into engagement with the rotary drive structure 280 of the bone screw head portion 24, the surgeon may turn the sleeve 508 in direction 520 about the shaft 506 to cause a radially enlarged portion 570 of the sleeve 508 to rotate into contact with the slider 20 and shift the slider 20 in direction 100 from the interference position to the clearance position.

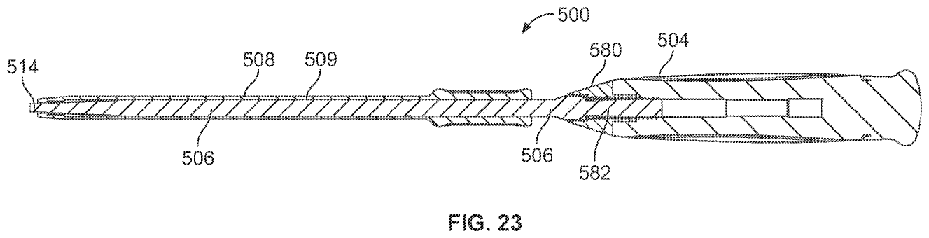

Regarding FIG. 23, a cross section of the bone screw remover tool 500 is provided. The handle 504 may be secured using adhesive, welds, or fastener(s) to a collar 580. The collar 580 may be threadingly engaged with a threaded portion 582 of the shaft 506. The shaft 506 extends through the cannula 509 of the sleeve 508 to project outward of the driving tip 514.

Regarding FIG. 24, the bone screw remover tool 500 has been connected to the bone screw 14 by aligning the recess 550 of the sleeve 508 with the slider 20 and advancing the driving tip 514 in direction 600 into the rotary drive structure 280 of the bone screw head portion 24. A radially narrow portion 569 of the sleeve 508 is positioned radially intermediate the slider 20 and the shaft 506. Next, the surgeon turns the sleeve 508 around the shaft 506 and cammingly engage the cam surface 560 of the sleeve 508 with the surface 610 of the slider 20 to shift the slider 20 from the interference position to the clearance position. The surgeon turns the sleeve 508 in direction 520 until the radially enlarged portion 570 of the sleeve 508 is radially intermediate the slider 20 and the shaft 506. The presence of the thicker, radially enlarged portion 570 against the slider 20 keeps the slider 20 in the clearance position. With the slider 20 in the clearance position, the surgeon may turn the handle 504 in direction 530 which causes the corresponding turning of the driving tip 514 in direction 530 and removes the bone screw 14 from the bone and the throughbore 518. The radially enlarged portion 570 of the sleeve 508 is sized to have an outer radius that is similar to a maximum outer radius of the bone screw head portion 24, such as a corner 620 (see FIG. 16), so that the radially enlarged portion 570 of the sleeve 508 may hold the slider 20 in the clearance position as the head portion 24 is removed in direction 601 until the slider 20 can transition into contact with the tapered surface 260 of the bone screw head portion 24. The slider 20 may slide along the tapered surface 26 as the bone screw head portion 24 is removed from the throughbore 18.

Uses of singular terms such as “a,” “an,” are intended to cover both the singular and the plural, unless otherwise indicated herein or clearly contradicted by context. The terms “comprising,” “having,” “including,” and “containing” are to be construed as open-ended terms. It is intended that the phrase “at least one of” as used herein be interpreted in the disjunctive sense. For example, the phrase “at least one of A and B” is intended to encompass A, B, or both A and B.

While there have been illustrated and described particular embodiments of the present invention, it will be appreciated that numerous changes and modifications will occur to those skilled in the art, and it is intended for the present invention to cover all those changes and modifications which fall within the scope of the appended claims.

1-29. (canceled)

30. A method of assembling a bone plate, the method comprising:
positioning a slider and a resilient member in a throughbore of a bone plate body;
advancing the slider and resilient member toward a receptacle of the bone plate body for the slider;
engaging opposite side portions of the resilient member with retainer portions of the bone plate body to urge the side portions of the resilient retainer together and deform the resilient member as the slider advances into the receptacle of the bone plate body; and
permitting the side portions of the resilient member to resiliently shift apart upon the side portions being advanced beyond the retainer portions of the bone plate body.

31. The method of claim 30 wherein positioning the slider and the resilient member in the throughbore includes positioning the slider and the resilient member in the throughbore with the resilient member extending in a through opening of the slider.

32. The method of claim 30 wherein engaging the opposite side portions of the resilient member with the retainer portions of the bone plate body includes sliding the opposite side portions of the resilient member along tapered surfaces of the retainer portions of the bone plate body.

33. The method of claim 30 wherein permitting the side portions of the resilient member to resiliently shift apart upon the side portions being advanced beyond the retainer portions of the bone plate body includes permitting the side portions to snap into recesses of the bone plate body.

34. The method of claim 30 wherein the resilient member includes a pin having opposite end portions and an intermediate portion; and
wherein engaging the opposite side portions of the resilient member with the retainer portions of the bone plate body to urge the side portions of the resilient retainer together includes engaging surfaces of the pin end portions with the retainer portions of the bone plate body while pressing a surface of the slider against the intermediate portion of the pin.
